(19) United States
(12) Patent Application Publication
(10) Pub. No.: US 2025/0213887 A1
(43) Pub. Date: Jul. 3, 2025

(54) OVERALL ABLATION WORKFLOW SYSTEM

(71) Applicant: Vektor Medical, Inc., Carlsbad, CA (US)

(72) Inventors: Christopher J. T. Villongco, Roswell, GA (US); Robert Joseph Krummen, Bellevue, WA (US)

(21) Appl. No.: 18/844,018

(22) PCT Filed: Mar. 3, 2023
(86) PCT No.: PCT/US2023/014406

§ 371 Date: Sep. 4, 2024

Related U.S. Application Data
(60) Provisional application No. 63/316,960, filed on Mar. 4, 2022.

(51) Int. Cl. A61N 5/10; G06T 7/00; G16H 50/70

(52) U.S. Cl. CPC A61N 5/1031; G06T 7/0012; G06T 2207/10081; G06T 2207/10088; G06T 2207/20081; G06T 2207/30048; G16H 50/70

(57) ABSTRACT

A technology is provided for supporting cardiac stereotactic ablative radiotherapy (SABR) procedure. The technology collects an arrhythmia electrocardiogram (ECG) from the patient and a CT scan. The technology employs a mapping system to generate a demarcated generic three-dimensional (3D) mesh based on the ECG. The demarcated generic 3D mesh has a target for an ablation demarcated. The technology employs a 3D machine learning (ML) model to generate a patient-specific 3D mesh based on the CT scan. The technology employs a demarcation ML model to generate a demarcated patient-specific 3D mesh based on the patient-specific 3D mesh and the demarcated generic 3D mesh. The demarcated patient-specific 3D mesh has the target for the ablation demarcated to account for difference between cardiac geometry of the patient-specific 3D mesh and the demarcated generic 3D mesh.

### CROSS-REFERENCE TO RELATED APPLICATIONS

This application claims the benefit of U.S. Application No. 63/316,960, filed on Mar. 4, 2022, entitled “OVERALL ABLATION WORKFLOW SYSTEM,” which is hereby incorporated by reference.

### BACKGROUND

Many heart disorders can cause symptoms, morbidity (e.g., syncope or stroke), and mortality. Common heart disorders caused by arrhythmias include inappropriate sinus tachycardia (IST), ectopic atrial rhythm, junctional rhythm, ventricular escape rhythm, atrial fibrillation (AF), ventricular fibrillation (VF), focal atrial tachycardia (focal AT), atrial microreentry, ventricular tachycardia (VT), atrial flutter (AFL), premature ventricular complexes (PVCs), premature atrial complexes (PACs), atrioventricular nodal reentrant tachycardia (AVNRT), atrioventricular reentrant tachycardia (AVRT), permanent junctional reciprocating tachycardia (PJRT), and junctional tachycardia (JT). The sources of arrhythmias may include electrical rotors (e.g., ventricular fibrillation), recurring electrical focal sources (e.g., atrial tachycardia), anatomically based reentry (e.g., ventricular tachycardia), and so on. These sources are important drivers of sustained or clinically significant arrhythmia episodes. Arrhythmias can be treated with ablation using different technologies, including radiofrequency energy ablation, other electromagnetic energy ablation, cryoablation, ultrasound ablation, laser ablation, external radiation sources, directed gene therapy, and so on by targeting the source of the heart disorder. Since the sources of heart disorders and the locations of the source vary from patient to patient, even for common heart disorders, targeted therapies require the source of the arrhythmia to be identified individually for each patient.

One technology that supports treating patients with ablations is stereotactic ablative radiotherapy (SABR), also known as stereotactic body radiation therapy (SBRT). SABR is a highly focused radiation treatment that administers a high-intensity dose of radiation concentrated at a target region of an organ (e.g., heart or lung) while limiting the dose to the surrounding organs. The intense dose is administered in a series of low-intensity doses administered from different angles relative to the target region. A delivery angle may be specified by the orientation of a delivery arm. The goal of applying low-intensity doses at different angles is to reduce the chance of healthy tissue being damaged while the cumulative application of low-intensity doses focused on the target region results in the application of a high-intensity dose to the target region that is sufficient to terminate electrical activity of faulty tissue in the target region.

A SABR device includes a radiation delivery component mounted on a robotic manipulator, a scanning device (e.g., a CT scanner), and a guidance system. The guidance system guides the delivery of radiation during a procedure based on a delivery plan developed in part based on images of the patient collected during treatment of a patient. The delivery plan includes a 3D image (e.g., 2D slices) of the organ (e.g., heart) and surrounding structures (e.g., lungs) with the target region of the organ for the ablation and structures to avoid (avoidance structures) demarcated on the 3D image. The delivery plan also specifies the high-intensity dose to be applied. The SABR device determines the angles of delivery for each low-intensity dose. The guidance system guides the robot manipulator to positions at the desired angles based on CT scans collected during the procedure and factors in motion of the organ and structures (e.g., due to breathing). Once positioned at an angle, the SABR device delivers the specified low-intensity dose for that angle.

Unfortunately, a significant amount of time is currently needed for a medical provider, such as an electrophysiologist (EP), to develop a delivery plan for an SABR procedure on a patient. To perform an ablation on a heart to treat an arrhythmia, the medical provider needs to first identify the source location of the ablation. The source location has traditionally been identified by first collecting an arrhythmia electrocardiogram (ECG) of the patient during an arrhythmia episode. The medical provider then invasively paces the heart at various locations while collecting images and pacing ECGs. If a pacing ECG matches the arrhythmia ECG, then the medical provider may assume that the pacing location is the source location of the arrhythmia. The pacing location is then typically manually determined from the images collected while pacing at the pacing location. The medical provider may alternatively identify the source location based on analysis of CT scans collected during an arrhythmia episode.

The medical provider also collects a CT scan of the patient using a stand-alone CT scanner. The medical provider then manually demarcates on the CT scan the source location as the target region for the ablation to generate a demarcated CT scan. The medical provider also demarcates on the CT scan the avoidance structures. Since this is a manual process, the demarcated CT scan may not have the desired accuracy. For example, if an avoidance structure is not accurately demarcated, the ablation procedure may not have the desired result.

At the start of the ablation procedure, a SABR CT scan is collected from the patient using a CT scanner that is part of the SABR device. A medical provider, who may be different from the medical provider who generated the demarcated CT scan, then transfers the demarcations from the demarcated CT scan to the SABR CT scan to generate a demarcated SABR CT scan. Typically, a SABR CT scan has a lower resolution than the demarcated CT scan because the stand-alone CT scanner of a medical facility typically has a higher resolution than a SABR CT scanner. As result, the transfer of the demarcations needs to factor in the different resolutions. Again, since this is a manual process, the demarcated SABR CT scan may not have the desired accuracy.

The medical provider relies on the SABR device to develop the delivery plan for delivery angles and the low-intensity doses to be delivered at each angle based on the demarcated SABR CT scan and then executes the delivery plan. The SABR device, however, does not allow a delivery plan to be developed by the medical provider.

### BRIEF DESCRIPTION OF THE DRAWINGS

FIG. 1 is a flow diagram that illustrates the overall process of treating a patient using the OAW system in some embodiments.

FIG. 2 is a block diagram that illustrates components of the OAW system in some embodiments.

FIG. 3 is a flow diagram that illustrates the processing of a generate demarcated patient-specific 3D mesh component of the OAW system in some embodiments.

FIG. 4 is a flow diagram that illustrates the processing of a mapping system in some embodiments.

FIG. 5 is a flow diagram that illustrates the processing of the train demarcation machine learning model component of the OAW system in some embodiments.

FIG. 6 is a flow diagram that illustrates the process of a develop delivery plan component of the OAW system in some embodiment.

### DETAILED DESCRIPTION

Methods and systems are provided that support a treatment procedure for treating a patient with an arrhythmia or other medical condition to help ensure that the treatment is successful and that planning for the procedure is performed efficiently and accurately in less time than required by current treatment procedures. In some embodiments, an overall ablation workflow (OAW) system provides information to help inform an electrophysiologist (EP) in performing an ablation on a patient. The OAW system may employ a 3D machine learning (ML) model to generate a patient-specific 3D representation of all or a portion of the patient's heart and a labeling of segments within patient's heart and segments that are non-cardiac structures in the patient's thorax. The patient-specific 3D representation may be implemented as patient-specific 3D mesh with vertices, edges, and faces based on the patient's cardiac geometry. After the patient-specific 3D mesh is generated, the OAW system accesses a demarcated generic 3D mesh with cardiac characteristics (e.g., target location for an ablation) demarcated. The demarcated generic 3D mesh may be, for example, derived from a simulated 3D mesh based on a simulated cardiac geometry used in simulating electrical activity of a heart based on simulated cardiac characteristics (e.g., simulated cardiac geometry and simulated source location). The OAW system may employ a demarcation ML model that inputs the patient-specific 3D mesh and the demarcated generic 3D mesh and outputs a demarcated patient-specific 3D mesh with one or more cardiac characteristic demarcated. The demarcated patient-specific 3D mesh can then be provided to an ablation therapy device, displayed to a medical provider, employed to develop a delivery plan for the procedure, and so on to help inform treatment of the patient.

The OAW system is described primarily in the context of 3D images that are CT scans with CT slices. However, the OAW system may be used with any type of representation of a 3D image such as an MRI scan, a 3D image specified by a 3D mesh, a collection of 2D projections of a heart (or thorax), and so on. The OAW system is also described primarily in the context of an ablation performed on a heart. The OAW system may also be used to assist in ablations on or other treatments of a variety of organs (e.g., heart, lung, gastrointestinal tract, and brain) of a variety of patients (e.g., humans and animals) based on electrograms representing electromagnetic energy corresponding to abnormal electrical activity of those organs.

In some embodiments, the 3D ML model is trained using training data with training sets that each includes a CT scan of a heart labeled with a 3D mesh representing that heart. The training data may be generated, for example, by generating simulated 3D meshes based on various cardiac geometries and generating a simulated CT scan for each simulated 3D mesh. The simulated CT slices of the simulated CT scan may be generated by simulating a quantity of radiation emitted from a simulated radiation source that passes through a heart in the shape of the simulated 3D mesh onto a simulated CT slice. The quantity of radiation that impacts the simulated CT slice may be derived from attenuation coefficient based on matter types of cardiac and non-cardiac segments of a simulated thorax. Multiple CT scans may be generated based on a simulated 3D mesh that is each based on a different segmentation of a simulated thorax such as different cardiac orientations, lung size, and so on. To generate the simulated CT slices, the OAW system may employ a technique for generating simulated 2D images from a simulated 3D representation as described in U.S. Pat. No. 10,713,791 entitled “Computational Simulations of Anatomical Structures and Body Surface Electrode Positioning” and issued on Jul. 15, 2020 ('791 patent), which is hereby incorporate by reference. The 3D ML model may comprise ML sub-models such as a segmentation ML sub-model, a labeling ML sub-model, and a 3D ML sub-model. In such a case, each ML sub-model has its own training data and may be trained with separate or combined loss functions.

The segmentation ML sub-model inputs a CT slice and outputs a segmentation of the CT slice. A segment may be defined by the boundary of a segment region of the CT slice and optionally its constituent matter type. Since a CT slice may include non-cardiac structures (e.g., lungs, esophagus) within the thorax, the segmentation ML sub-model may also identify segments relating to such non-cardiac structures. However, a separate ML model may be used to segment non-cardiac structures. The constituent matter type of a segment may be air (e.g., within a lung), blood (e.g., within a ventricle or artery), cardiac tissue, bone tissue, and so on. The segments and constituent matter types may be specified using pixel coloring or shading. Alternatively, the segments and constituent matter types may be specified using metadata that specifies the boundaries of the segments and their constituent matter types. The segmentation ML sub-model may be trained using clinical or simulated data. The clinical data may be CT scans and corresponding segmented CT scans collected from electronic health records (EHRs), and the simulated data may be simulated CT scans and corresponding simulated segmented CT scans. The segmentation ML sub-model may be implemented using a variety of ML architectures such as a convolutional neural network (e.g., a U-Net architecture or other ML architectures as described below) that inputs a CT slice and outputs a segmented CT slice with its constituent matter types identified.

The labeling ML sub-model inputs a segmented CT slice(s) of a CT scan and outputs a labeling of the segments of the CT scan. The labels for the segments identify the thoracic structure (e.g., heart, esophagus and liver). In addition, for the heart, the labels identify cardiac segments such as right atrium, left ventricle, interventricular septum, tricuspid valve, pulmonary arteries and veins, other cardiac vessels, cardiac nerves, and so on. The labeling ML sub-model may be implemented using a variety of architectures such as a convolutional neural network that inputs a segmented CT slice and outputs a labeled CT slice. The labeling ML sub-model may be trained using clinical or simulated data. The clinical data may be segmented CT scans and corresponding labeled CT scans collected from EHRs, and the simulated data may be simulated segmented CT scans and corresponding simulated labeled CT scans. In some embodiments, the segmentation ML sub-model and the labeling ML sub-model may be combined into a single ML sub-model that inputs a CT scan and outputs a labeling of the segments of the CT scan.

The 3D ML sub-model inputs a labeled CT scan and outputs a patient-specific 3D mesh for the patient. The 3D ML sub-model may be a recurrent convolutional neural network or a graph neural network that outputs coordinates for vertices of the patient-specific 3D mesh as the labeled CT slices are processed by the 3D ML sub-model.

Rather than employing the 3D ML model to generate a patient-specific 3D mesh from a CT scan or any one of its ML sub-models, the OAW system may employ other systems for generating a patient-specific 3D mesh. Such systems include 3D Slicer, OsiriX, and ITK-SNAP. For example, the OAW system may employ 3D Slicer to convert the output of the labeling ML sub-model to the patient-specific 3D mesh. A variety of segmentation techniques are described in Wirjdai, O., “Survey of 3D Image Segmentation Methods,” Model and Algorithms in Image Processing, Fraunhofer ITWM, Kaiserslautern, Germany, 2007, which is hereby incorporated by reference.

The OAW system may identify the cardiac characteristic(s) using a patient arrhythmia cardiogram collected from a patient during an arrhythmia episode. A cardiogram may be, for example, an electrocardiogram (ECG) or a vectorcardiogram (VCG). The OAW system then provides the patient arrhythmia cardiogram to a mapping system that identifies a simulation with simulated cardiac characteristics that would generate a simulated arrhythmia cardiogram similar to the patient arrhythmia cardiogram. The mapping system may generate demarcated generic 3D meshes based on simulations of electrical activity of a heart assuming different cardiac characteristics such as source location of an arrhythmia, scar tissue location, cardiac geometry and orientation, electrical characteristics, and so on. A technique for simulating electrical activity of a heart is described in U.S. Pat. No. 10,860,754 entitled “Calibration of Simulated Cardiograms” and issued on Dec. 8, 2020 ('754 patent), which is hereby incorporated by reference. The mapping system may generate the demarcated generic 3D meshes in advance or on-demand based on cardiac geometry associated with the identified simulation. In addition, since many simulations may be based on the same simulated cardiac geometry, the mapping system may have a pre-generated generic 3D mesh for each simulated cardiac geometry rather than one for each simulation. In such a case, the mapping system generates a demarcated generic 3D mesh on demand based on a generic 3D mesh and simulated cardiac characteristics of the identified simulation. A generic 3D mesh is “generic” in the sense that it is not generated based on the patient's cardiac geometry. However, the OAW system may employ the calibration techniques of the '754 patent. In such a case, the generic 3D mesh may be considered somewhat specific to the patient. Rather than or in addition to using simulated 3D meshes, the mapping system may be based on clinical data collected from EHRs with cardiac geometry data and other cardiac characteristics from which demarcated generic 3D meshes can be generated.

After identifying a demarcated generic 3D mesh with one or more regions of interest (ROIs) demarcated, the OAW system inputs the patient-specific 3D mesh and the demarcated generic 3D mesh to the demarcation ML model to generate a demarcated patient-specification 3D mesh. The ROIs may be, for example, a simulated source location of an arrhythmia and region of simulated scar tissue. As another example, an ROI may be a region defining an ablation pattern for the ablation to be performed. Techniques for identifying an ablation pattern are described in U.S. Pat. No. 11,259,871 entitled “Identify Ablation Pattern for Use in an Ablation” and issued on Mar. 1, 2022 ('871 patent), which is hereby incorporated by reference. The OAW system may specify the ROIs of a demarcated patient-specific 3D mesh using pixel coloring or shading or using metadata defining the ROIs. The demarcation ML model may employ a neural network that inputs the patient-specific 3D mesh and the demarcated generic 3D mesh and outputs the demarcated patient-specific 3D mesh. The demarcated patient-specific 3D mesh represents a heart that is based on the patient's cardiac geometry with ROIs (e.g., arrhythmia source location) positioned factoring in differences between the patient's cardiac geometry and the generic cardiac geometry. Rather than employing machine learning, the OAW system may generate a transformation matrix to transform vertices of the demarcated generic 3D mesh to corresponding vertices of the patient-specific 3D mesh and then apply the transformation matrix to the ROIs to transform them to the patient-specific 3D mesh. The demarcated patient-specific 3D mesh may be converted to a Digital Imaging and Communications in Medicine (DICOM) format and then may be provided to a treatment device to help inform the treatment.

In some embodiments, the OAW system may provide the demarcated patient-specific 3D mesh or the information content of it in another format (e.g., CT scan) to a SABR device or other treatment device for use in planning the patient's treatment. For example, the OAW system may provide an ablation therapy device with a demarcated patient-specific CT scan with simulated CT slices derived from a slicing of the demarcated patient-specific 3D mesh as described in the '791 patent. The OAW system generates the demarcated patient-specific CT scan in the resolution of a scanning device integrated with the SABR device which may be a lower resolution than a typical scanning device of a medical facility. The demarcated patient-specific CT scan also includes a demarcation of the non-cardiac structures that may be identified by the segmentation ML sub-model.

The ablation therapy device may generate a delivery plan for the treatment based on the demarcated patient-specific CT scan or the demarcated patient-specific 3D mesh. Alternatively, the OAW system may generate the delivery plan and provide it to an ablation therapy device adapted to receive such delivery plans. To generate the delivery plan, the OAW system identifies avoidance segments (e.g., nerves and esophagus) based on the labeling of the segments. The OAW system then applies a planning ML model to the demarcated patient-specific CT scan (or 3D mesh) and avoidance segments indicated and radiation dosage information to generate the delivery plan. The delivery plan specifies the movement of the robotic manipulator, portion of the dose to be delivered at each orientation of a delivery arm, shape of a delivery beam, and so on. The procedure can then be performed by the ablation therapy device in accordance with the delivery plan.

In some embodiments, the mapping system compares a patient arrhythmia cardiogram to a library of library cardiograms that are each associated with a demarcated generic 3D mesh based on simulated cardiac characteristics. The library cardiograms may be generated based on simulated electrical activity of hearts with different simulated cardiac characteristics (or heart data) such as different cardiac geometries, electrical properties, scar locations, source locations, prior ablation locations, prior or suggested ablation patterns, and so on. Each library cardiogram is associated with the simulated cardiac characteristics used in the simulation from which the library cardiogram was generated. The source location associated with a library cardiogram that is similar to the patient arrhythmia cardiogram (e.g., based on a similarity or matching metric generated using a Pearson correlation technique or other similarity criterion) may represent the source location of the patient's arrhythmia and thus the target region for the ablation. The ablation pattern used in a simulation to terminate the arrythmia (see '781 patent) may also be the ablation pattern to be used in the ablation procedure on the patient. To identify a similar library cardiogram, the mapping system may first apply a filter to the library (e.g., calibrate the library) to identify library cardiograms that have simulated cardiac characteristics (e.g., cardiac geometry or conduction velocity) similar to the patient's cardiac characteristics. (See, patient matching and calibration of the '754 patent.) The mapping system then identifies a similar library cardiogram from the filtered library that is similar to the patient cardiogram. A demarcated generic 3D mesh is generated based on cardiac characteristics (e.g., geometry and orientation) associated with a similar library cardiogram. The demarcated generic 3D mesh may be generated in advance of a simulation, after a simulation, or on demand when planning a procedure.

The OAW system may alternatively employ a mapping system that identifies the source location of an arrhythmia by applying a mapping ML model to an arrhythmia cardiogram (and possibly some patient cardiac characteristics) which outputs a source location and possibly other cardiac characteristics or ROIs. The mapping ML model may be trained using the library cardiograms labeled with their associated source locations and possibly labeled with simulated characteristics such as a simulated cardiac geometry. (See, the '754 patent for an example of a mapping ML model.) The mapping ML model may also be trained using demarcated generic 3D meshes as a label so that the mapping ML model also outputs a demarcated generic 3D mesh. Prior to training, the library cardiograms may be calibrated to patient characteristics of the patient's heart by identifying a subset of the library cardiograms that have similar cardiac characteristics (filtering) and training the mapping ML model based on that subset. In such a case, the mapping ML model may be trained during procedure planning using techniques as described in the '754 patent such as transference of model weights. In some embodiments, the OAW system may also identify an ablation pattern for the ablation using the ablation pattern identification (API) system as described in the '871 patent.

In some embodiments, the OAW system generates a patient-specific 3D graphic based on the demarcated patient-specific 3D mesh and displays the patient-specific 3D graphic with an indication of one or more source locations (i.e., as volumes) superimposed on the heart. A system for generating such a graphic, referred to as a source location (SL) graphic, is described in U.S. Pat. No. 10,709,347, entitled “Heart Graphic Display System,” and issued on Jul. 14, 2020 ('347 patent), which is hereby incorporated by reference. The '347 patent describes identifying one or more source locations using a mapping system such as described above. The source locations may be represented by various indicators such as an X marking a source location, color variations such as variations in intensities to distinguish likely source locations from less likely source locations, and so on.

In some embodiments, the OAW system may generate a demarcated patient-specific CT scan by projecting the demarcated patient-specific 3D mesh onto the patient's CT scan collected from the patient with ROIs indicated by the mapping system demarcated. The OAW system may employ a projection algorithm that, for each 2D slice of the patient's CT scan, identifies a corresponding 2D slice of the demarcated patient-specific 3D mesh. The OAW system then demarcates the 2D slice of the patient's CT scan based on the ROIs of the corresponding 2D slice of the demarcated patient-specific 3D mesh.

The OAW system may alternatively employ a projection ML model to generate a demarcated patient-specific CT scan. To train the projection ML model, the OAW system accesses training data with training sets that each includes a CT scan of a heart and a demarcated 3D mesh of that heart that are labeled with a demarcated CT scan with ROIs demarcated. The OAW system may generate a feature vector and a label for each training set. A feature vector includes one or more features derived from the CT scan and the demarcated 3D mesh and the label may be the demarcated CT scan itself. For example, the features of a CT scan may be the CT scan itself, segments of the CT scan, features based on a principal component analysis, and so on. The OAW system then trains the projection ML using the feature vectors and labels. The OAW system uses the projection machine ML model to generate a demarcated patient-specific CT scan given a CT scan of a patient and a demarcated patient-specific 3D mesh. The training data may be derived from CT scans and demarcated CT scans developed for procedures that did not employ the OAW system.

The projection ML model may be based on various ML architectures. For example, the projection ML model may include a convolutional neural network for the patient-specific 3D mesh and a convolutional neural network for the CT scan. The outputs of the convolutional neural networks are then input to a fully connected layer which outputs the demarcated patient-specific CT scan. The loss function may be a combined loss function of the convolutional neural networks and the fully connected layer which are trained jointly. A different ML architecture may be employed if the demarcations are represented as metadata. In such a case, in addition to the two convolutional neural networks mentioned above, a separate neural network may be employed that inputs the metadata and generates output that is provided to the fully connected layer. Also, rather than employing convolutional neural networks, a neural network may input features derived from demarcated patient-specific 3D mesh and the CT scan and output the demarcated patient-specific CT scan. As another example, an autoencoder may be trained using the demarcated 3D meshes, and another autoencoder may be trained using the CT scans. A neural network can then be trained using the latent vectors of the autoencoders labeled with a demarcated CT scan. Alternatively, the autoencoders and the neural networks may be trained jointly. As another alternative, an autoencoder may be trained using the demarcated CT scans and the labels for the neural network may be the corresponding latent vectors. To generate a demarcated CT scan, the autoencoders are employed to generate latent vectors for a 3D mesh and CT scan, the latent vectors are input to the neural network to generate a latent vector representing a demarcated CT scan, and that latent vector representing the demarcated CT mesh are input into the corresponding autoencoder which outputs the demarcated 3D image. A convolutional neural network that inputs a CT scan or other 3D image may apply a 3D convolutional window so that an activation function represents pixels in the same CT slice but also pixels in adjacent CT slices. These ML architectures may be employed in the various ML models and ML sub-models described above.

In some embodiments, the demarcated patient-specific CT scans and planning CT scans of the ablation therapy may have different resolutions such as different pixel resolutions (e.g., 4K by 4K v. 2K by 2K) and different numbers of slices (e.g., 1024 v. 512). A transfer algorithm may be employed to set the pixel value of a pixel (e.g., representing a voxel) for a target pixel location in 3D space of the demarcated planning CT scan based on the pixel values of pixels of the demarcated patient-specific CT scan with pixel locations in a neighborhood of that target pixel location. For example, the pixel value for the designated location may be set to an average of the pixel values of pixels in the neighborhood.

In some embodiments, the OAW system may train an avoidance ML model to generate an avoidance demarcated (planning) CT scan with target regions and avoidance regions demarcated. (The avoidance regions may alternatively be identified by coordinates in a 3D space relative to the position of the heart.) The OAW system accesses training data with training sets that include a CT scan, a target region, and an avoidance demarcated CT scan. The OAW system generates a feature vector for each training set. A feature vector includes one or more features derived from the CT scan (e.g., the CT scan itself or a latent vector) and one or more features derived from the target region, and the label is derived from the avoidance demarcated CT scan (e.g., the demarcate CT scan itself or a latent vector). The OAW system trains the avoidance ML model using the feature vectors and labels. The avoidance ML model may employ the segmentation ML sub-model and the labeling ML sub-model as described above. The OAW system uses the avoidance ML model to generate an avoidance demarcated CT scan given a CT scan and a target region. In some embodiments, the avoidance ML model may be trained to output indications of the avoidance regions relative to the CT scan. Thus, the avoidance demarcated CT scan may be considered to be the combination of the CT scan and metadata (e.g., annotations or tags) indicating the avoidance regions. The training data may be derived from CT scans, target regions, and avoidance demarcated CT scans developed in procedures that did not employ the OAW system. Various ML architectures may be used for the avoidance machine learning model such as those describe above.

In some embodiments, the OAW system may train a planning ML model to generate a delivery plan for an ablation procedure. The OAW system accesses training data with training sets that include an avoidance demarcated CT scan with a demarcation of the target region (and possibly an ablation pattern) and other non-cardiac structures and avoidance regions, a target dose to be delivered to the target region, a maximum dose for non-target regions, and a delivery plan (include, for example, angles of delivery, low-intensity dose for each angle, arcs of movement of a robotic manipulator, shape of delivery beam). The OAW system generates a feature vector and label for each training set. A feature vector includes features derived from the avoidance demarcated CT scan (e.g., indicating geometry and position of avoidance regions and a target region), a target dose, maximum dose for non-target regions, and so on and the label is derived from the delivery plan. The OAW system trains the planning ML model using the feature vectors and labels. The OAW system uses the planning ML model to generate a delivery plan given an avoidance demarcated CT scan with an indication of the target region and dosage information. The training data may be derived from avoidance demarcated CT scan, doses, and delivery plans developed in procedures that did not employ the OAW system. In some embodiments, the avoidance demarcated CT scans used for training may be generated from CT scan collected from patients or simulation CT scan used in simulation of the mapping system.

In some embodiments, the OAW system may identify possible angles of delivery based on the avoidance structures. The OAW system may employ a planning algorithm to identify the angles of delivery (i.e., supported by the ablation therapy device) that intersect the target region and do not intersect an avoidance structure. Such angles are considered to be candidate angles. The planning algorithm may generate a delivery plan by selecting delivery angles from the candidate angles that do not intersect other candidate angles outside of the target region and specifying an angle dose for each delivery angle. The angle dose for a delivery angle that may be the target dose divided by the number of delivery angles. The dose applied to a target sub-region of the target region is the total of the angle doses of the delivery angles that intersect that target sub-region. The OAW system may employ a greedy algorithm that processes the target sub-regions in sequence by, for each target sub-region, selecting delivery angles factoring in the number of delivery angles selected for other target sub-regions and maximum dose for a target sub-region given the selected delivery angles. If a maximum dose is exceeded for any target sub-region, another set of delivery angles is selected for one or more sub-regions. If an angle dose can be variable, then the planning algorithm would also vary the angle doses as part of the greedy algorithm. Although the greedy algorithm may not find a delivery plan that is optimal (e.g., least number of angles), the delivery plan found would deliver the target dose to each sub-region.

In some embodiments, the OAW system allows a medical provider to revise the demarcations, the delivery plan, and so on as the medical provider deems appropriate. For example, a patient may have a medical or physical condition that results in a segment that is not an accurate representation of the corresponding avoidance regions (e.g., too small or misshaped) or that has its avoidance status incorrectly designated. Even with such revisions, the use of the OAW system has significant advantages over current techniques such as speed, in most cases accuracy, and may be more comprehensive because it may identify and factor in aspects that a medical provider may not.

A machine learning (ML) models of the OAW system may be any of a variety or combination of supervised, semi-supervised, self-supervised, or unsupervised ML models including a neural network such as fully connected, convolutional, recurrent, or autoencoder neural network, or restricted Boltzmann machine, a support vector machine, a Bayesian classifier, k-means clustering, decision tree, generative adversarial networks, and so on. When the ML model is a deep neural network, the model is trained using training data that includes features derived from data and labels corresponding to the data. For example, the data may be images of animals and the labels may be the names of the animals. The training results in a set of weights for the activation functions of the layers of the deep neural network. The trained deep neural network can then be applied to new data to generate a label for that new data. When the ML model is a support vector machine, a hyper-surface is found to divide the space of possible inputs. For example, the hyper-surface attempts to split the positive examples (e.g., real images represented by features) from the negative examples (e.g., fake images represented by features) by maximizing the distance between the nearest of the positive and negative examples to the hyper-surface. The trained support vector machine can then be applied to new data to generate a classification (e.g., real or fake) for the new data. A ML model may generate values of discrete domain (e.g., classification), probabilities, and/or values of a continuous domain (e.g., regression value, classification probability).

Various techniques can be used to train a support vector machine such as adaptive boosting, which is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates increasingly on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, loss function, and search algorithm. The architecture defines the functional form relating the inputs to the outputs (in terms of network topology, unit connectivity, and activation functions). The search in weight space for a set of weights that minimizes the loss function is the training process. A neural network model may use a radial basis function (RBF) network and a standard or stochastic gradient descent as the search technique with backpropagation.

A convolutional neural network (CNN) has multiple layers such as a convolutional layer, a pooling layer, a fully connected (FC) layer, and so on. Some more complex CNNs may have multiple convolutional layers, pooling layers, and FC layers. Each layer includes a neuron for each output of the layer. A neuron inputs outputs of prior layers (or original input) and applies an activation function to the inputs to generate an output.

A convolutional layer may include multiple filters (also referred to as kernels or activation functions). A filter inputs a convolutional window, for example, of an image, applies weights to each pixel of the convolutional window, and outputs value for that convolutional window. For example, if the static image is 256 by 256 pixels, the convolutional window may be 8 by 8 pixels. The filter may apply a different weight to each of the 64 pixels in a convolutional window to generate the value.

An activation function has a weight for each input and generates an output by combining the inputs based on the weights. The activation function may be a rectified linear unit (ReLU) that sums the values of each input times its weight to generate a weighted value and outputs max(0, weighted value) to ensure that the output is not negative. The weights of the activation functions are learned when training a ML model. The ReLU function of max(0, weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

An FC layer in which each of the neurons inputs all the outputs of the prior layer and generates a weighted combination of those inputs. For example, if the penultimate layer generates 256 outputs and the FC layer inputs a neuron for each of three classifications (e.g., dog, cat, and bird), each neuron inputs the 256 outputs and applies weights to generate value for its classification.

A graph neural network (GNN) is designed to operate on graph data based on convolutions over neighborhoods of nodes of the graph data. (See, Gori, M., et al., “A New Model for Learning in Graph Domains,” Proc. 2005 IEEE Int. Joint Conf. on Neural Networks, 2005 (vol. 2, pp. 729-734) IEEE, which is hereby incorporated by reference.) The training data may include graphs with each node having features and a label. When training a GNN, nodes send information to neighboring nodes based on a loss function to learn the weights for features of the nodes. After the GNN is trained, graph data can be input to the GNN to generate a label for each node based on the features of the nodes of the graph data.

A generative adversarial network (GAN) or an attribute (attGAN) may also be used. An attGAN employs a GAN to train a generator model. (See, Zhenliang He, Wangmeng Zuo, Meina Kan, Shiguang Shan, and Xilin Chen, “AttGAN: Facial Attribute Editing by Only Changing What You Want,” IEEE Transactions on Image Processing, 2019; and lan Goodfellow, Jean Pouget-Abadie, Mehdi Mirza, Bing Xu, David Warde-Farley, Sherjil Ozair, Aaron Courville, and Yoshua Bengio, “Generative Adversarial Nets,” Advances in Neural Information Processing Systems, pp. 2672-2680, 2014, which are hereby incorporated by reference.) An attGAN includes a generator and discriminator and an attGAN classifier and is trained using training data that includes input images of objects and input attribute values of each object. The generator includes a generator encoder and a generator decoder. The generator encoder inputs an input image and is trained to generate a latent vector of latent variables representing the input image. The generator decoder inputs the latent vector for an input image and the input attribute values. The attGAN classifier inputs an image and generates a prediction of its attribute values. The attGAN is trained to generate a modified image that represents the input image modified based on the attribute values. The generator encoder and the generator decoder form the generator model.

Multimodal machine learning combines different modalities of input data to make a prediction. The modalities may be, for example, images, text, and audio.

In one multimodal machine learning approach, referred to as “early fusion,” data of the different modalities is combined at the input stage and is then trained on the multimodal data. The training data for these modalities include a collection of sets of an image, related text, and related audio and labels. The image, text, and audio may be used in its original form or preprocessed, for example, to reduce its dimensionality by compressing the data into byte arrays or applying a principal component analysis. Also, the resolutions of the image and audio may be reduced. The concatenated bytes may be then processed by a cross-attention mechanism to condense the concatenated bytes into a vector of a fixed size. The vectors are then used to train an ML model using primarily using supervised approaches although self-supervised or unsupervised approaches may also be used.

In a second multimodal machine learning approach, data from different modalities may be kept separate at the input stage and used as inputs to different, modality-specific ML models (e.g., a CNN for image data and a recurrent neural network (RNN) for sequential data). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label.

Transformer machine learning employs an attention mechanism to process tokens representing the input in parallel. For example, if a transformer ML model is used to process a sentence, each word may be represented as a token that includes an embedding of a word and its positional information. The embedding is a vector representation of a word such that words with similar meaning are closer in the vector space. The positional information is based on position of the word in the sentence.

A transformer ML model includes encoders with a neural network and self-attention mechanism and decoders with an attention mechanism, a self-attention mechanism, and a neural network.

The first layer of the encoder inputs the tokens, and each other layer inputs the encodings of the previous layer. The self-attention mechanism inputs the encodings of the previous layer (or tokens for the first encoder) and weighs the relevance of each encoding to each other to generate input for the neural network which generates the encodings.

The first layer of decoder inputs data based on the tokens, and each other layer inputs the decoding of the prior layer. Each layer of the decoder also inputs the encodings of the encoder. The attention and self-attention mechanisms input the decodings (or tokens for the first layer) and weight the relevance of each decoding to each other to generate input for the neural network.

Although initially developed to process sentences, transformers have been adapted for image recognition. The input to the transformer is a representation of fixed-size patches of the image. The representation of a patch may be, for each pixel of the patch, an encoding of its row, column, and color. The output of the transformer may be, for example, a classification of the image.

Fine-tuning learning is an ML technique to train an ML model by leveraging a previously trained ML model. For example, if a cat/tiger ML model has been trained to recognize whether an image has a cat, a tiger, or neither, fine tuning may be used to train a cat/tiger breed ML model to recognize whether an image has a certain breed of cat (e.g., Toyger), a certain breed of tiger (e.g., Bengal), or neither. The inner layers of the cat/tiger breed ML model have weights that are the same as the inner layers of the cat/tiger ML model. The input layer and the output layer of the cat/tiger breed ML model are trained using images labeled as that cat breed, that tiger breed, or neither. Thus, the training process need only learn the weights for the input layer and the output layer. In the cat and tiger case, the rationale for using fine tuning is that, since cats and tigers have someone similar feature, the weights of the inner layers of the cat/tiger ML model and cat/tiger breed ML model that are separately trained are similar. However, the input layer and the output layer of the cat/tiger breed ML model needs to be trained to account for the differences between a certain breed of cat and certain breed of tiger. Moreover, the fine tuning may be based on a smaller set of training data than used for training the cat/tiger ML model.

Self-supervised learning is an ML technique that is based on unlabeled training data. Initially, self-supervised learning augments the training data to generate additional training data to generate sets of training data that are similar. For example, if the training data is images of cats and tigers, the self-supervised learning generates for each image images of varied sizes, shading, and orientations. An ML model may have an encoder layer, pretext task layer, and contrastive learning. The encoder layer encodes the images into a latent vector. The pretext task layer includes weights for grouping images into different clusters based on their differences using contrastive learning. Contrastive learning employs a loss function for contrasting the images and adjusting the weights of the encoder and the pretext task layer. This approach is similar to k-means clustering but based on contrast rather than similarity. The weights of the pretext layer can be used as initial weights of a primary task such as a cat/tiger breed ML model which can be trained using labeled training data. Self-supervised learning may be performed on multimodal data as described above.

The computing systems on which the OAW system may be implemented may include a central processing unit, input devices, output devices (e.g., display devices and speakers), storage devices (e.g., memory and disk drives), network interfaces, graphics processing units, cellular radio link interfaces, global positioning system devices, and so on. The input devices may include keyboards, pointing devices, touch screens, gesture recognition devices (e.g., for air gestures), head and eye tracking devices, microphones for voice recognition, and so on. The computing systems may include desktop computers, laptops, tablets, e-readers, personal digital assistants, smartphones, gaming devices, servers, and so on. The computing systems may access computer-readable media that include computer-readable storage media (or mediums) and data transmission media. The computer-readable storage media are tangible storage means that do not include a transitory, propagating signal. Examples of computer-readable storage media include memory such as primary memory, cache memory, and secondary memory (e.g., DVD) and other storage. The computer-readable storage media may have recorded on it or may be encoded with computer-executable instructions or logic that implements the OAW system. The data transmission media is used for transmitting data via transitory, propagating signals or carrier waves (e.g., electromagnetism) via a wired or wireless connection. The computing systems may include a secure cryptoprocessor as part of a central processing unit for generating and securely storing keys and for encrypting and decrypting data using the keys. The computing systems may be servers that are housed in a data center such as a cloud-based data center.

The OAW system may be described in the general context of computer-executable instructions, such as program modules and components, executed by one or more computers, processors, or other devices. Generally, program modules or components include routines, programs, objects, data structures, and so on that perform particular task or implement particular data types. Typically, the functionality of the program modules may be combined or distributed as desired in various embodiments. Aspects of the OAW system may be implemented in hardware using, for example, an application-specific integrated circuit (“ASIC”) or field programmable gate array (“FPGA”).

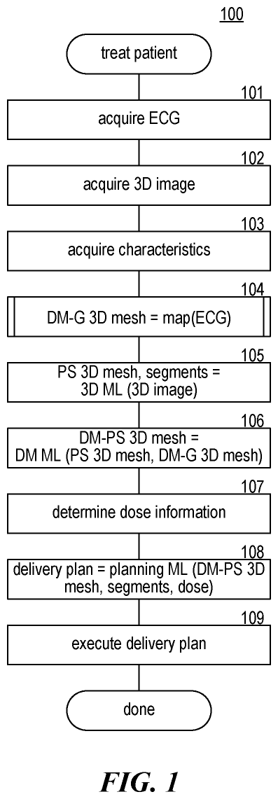

FIG. 1 is a flow diagram that illustrates the overall process of treating a patient using the OAW system in some embodiments. The treatment process collects data from the patient including a 3D image that is a CT scan of the patient's thorax, patient characteristics, and an arrhythmia ECG, employs a mapping system to generate a demarcated generic 3D mesh with a target region demarcated, employs a 3D ML model and a demarcation ML model to generate a demarcated patient-specific 3D mesh and labeling of structures (e.g., segments), and employs a planning ML model to generate a delivery plan based on the demarcated patient-specific 3D mesh and the labeling of the structures along with other information such as dosage information. In block 101, the process acquires an ECG from the patient. In block 102, the process acquires a CT scan of the patient's heart and other portions of the thorax. In block 103, the process acquires patient characteristics such as cardiac geometry, scar location, electrical characteristics, and so on. In block 104, the process applies a mapping system to the ECG to generate a demarcated (DM) generic (G) 3D mesh with a source location of an arrhythmia demarcated. The mapping system may convert the ECG to a VCG when the mapping system employs cardiograms that are VCGs. In block 105, the process applies a 3D ML model to the CT scan to generate a patient-specific (PS) 3D mesh and a segmentation of the CT scan. In block 106, the process applies a demarcation ML model to the patient-specific 3D mesh and the demarcated generic 3D mesh to generate a demarcated patient-specific 3D mesh. In block 107, the process determines a dose of radiation to apply to a target region of the heart. In block 108, the process applies a planning ML model to the demarcated patient-specific 3D mesh, segmentation, and dose information to generate the delivery plan. In block 109, the process directs execution of the delivery plan and then completes.

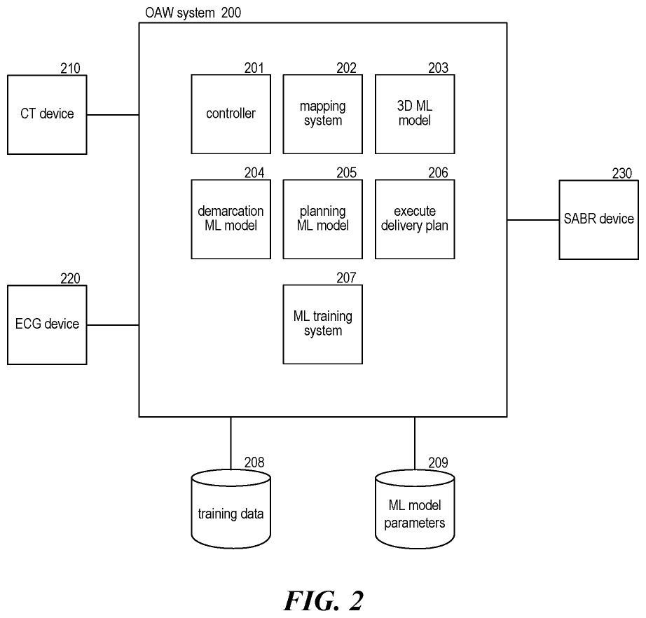

FIG. 2 is a block diagram that illustrates components of the OAW system in some embodiments. The OAW system 200 is connected to a CT device 210, an ECG device 220, and a SABR device 230. The OAW system includes a controller 201, a mapping system 202, a 3D ML model component 203, a demarcation ML model component 204, a planning ML model component 205, an execute delivery plan system 206, and an ML training system 207. The OAW system also includes a training data set store 208 and a model parameters store 209. The controller controls the overall processing of the OAW system. The controller collects an ECG of a patient from the ECG device and a CT scan of the patient from the CT device. The controller invokes the mapping system to generate a demarcated generic 3D mesh given the ECG. The controller employs the 3D ML model component to generate a patient-specific 3D mesh and a segmentation based on the CT scan. The controller employs the demarcation ML model component to generate a demarcated patient-specific 3D mesh based on the demarcated generic 3D mesh and the patient-specific 3D mesh. Although not illustrated, the controller may generate a demarcated patient-specific CT scan based on the demarcated patient-specific 3D mesh. The controller employs the planning ML model component to generate a delivery plan based on the demarcated patient-specific 3D mesh, segmentation (e.g., indicating avoidance regions), radiation dosage information, characteristics of the SABR device (e.g., available delivery angles), and so on. The OAW system may employ a planning ML model that is specific to a type (e.g., manufacturer) of SABR device. For example, different types of SABR devices may require different types of information in a delivery plan and have different capabilities (e.g., delivery angles). One SABR device may expect an input delivery plan to include only target dose only and will calculate the dose for each delivery angle or receive dosage guidance from a technician during a procedure. Another SABR device may expect an input delivery plan to include the dose for each angle. The ML training system trains various ML models (described above) using the training data of the training data store to create model parameters (e.g., weights and biases) for each of the ML models and stores them in the model parameter store.

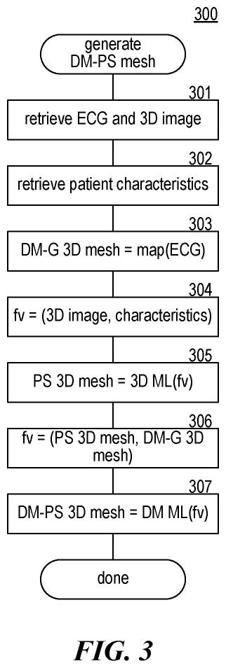

FIG. 3 is a flow diagram that illustrates the processing of a generate demarcated patient-specific 3D mesh component of the OAW system in some embodiments. The generate demarcated patient-specific 3D mesh component 300 collects patient data and employs a mapping system to generate a demarcated patient-specific 3D mesh. In block 301, the component retrieves an ECG and a 3D image (e.g., CT scan) of a patient. In block 302, the component retrieves patient characteristics such as cardiac geometry, scar locations, and prior ablation locations. In block 303, the component applies the mapping system to the ECG and optionally the patient characteristics to generate a demarcated generic 3D mesh. In block 304, the component generates a feature vector based on features derived from the 3D image and the patient characteristics. In block 305, the component applies the 3D ML model to the feature vector to generate a patient-specific 3D mesh. In block 306, the component generates a feature vector based on features derived from the patient-specific 3D mesh and the demarcated generic 3D mesh. In block 307, the component applies the demarcated ML model to the feature vector to generate a demarcated patient-specific 3D mesh and then completes.

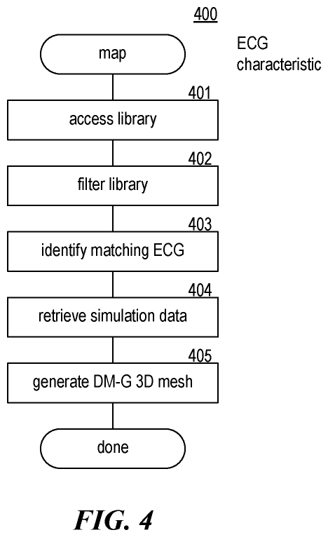

FIG. 4 is a flow diagram that illustrates the processing of a mapping system in some embodiments. The mapping system 400 is provided a patient ECG and patient characteristics and returns a demarcated generic 3D mesh. In block 401, the component accesses a library that maps each library cardiogram (e.g., VCG) to simulation data used in a simulation to generate the library cardiogram. The simulation data may include, for example, a simulated cardiac geometry of a heart used in the simulation and a simulation mesh with vertices corresponding to locations in the heart for which values of electrical characteristics are calculated for each simulation interval. In block 402, the component filters out library cardiograms associated with simulation characteristics that are not similar to the patient characteristics based on a similarity criterion. For example, the filtering may filter out library cardiograms associated with simulated characteristics (e.g., scar locations) that are very different from the patient characteristics. In block 403, the component identifies a library cardiogram that matches patient ECG. When the library cardiogram is a library VCG, the component may convert the patient ECG to a patient VCG and compare the library VCG to the patient VCG to determine if they satisfy a similarity criterion. In block 404, the component retrieves simulation data associated with matching library cardiogram. In block 405, the component generates a demarcated generic 3D mesh with ROIs demarcated based on the simulation data. For example, the demarcated generic 3D mesh may be generated from the simulated cardiac geometry or a simulation mesh. Alternatively, a demarcated generic 3D mesh may be generated for each simulation in advance and stored in the library mapped to the library cardiogram of that simulation. The component then completes.

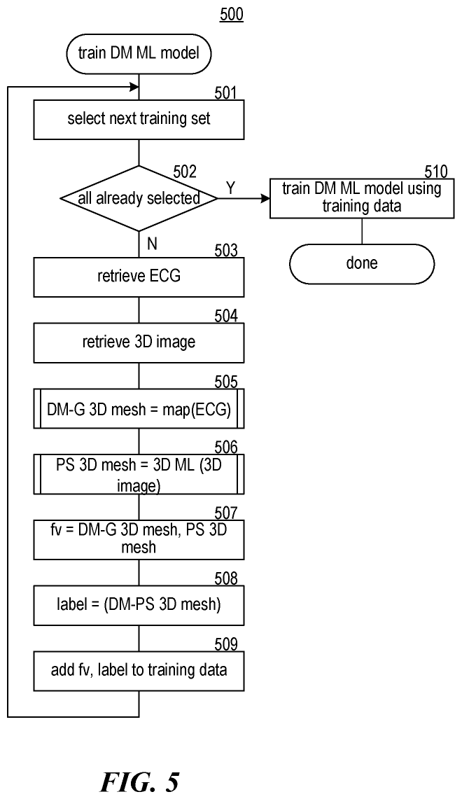

FIG. 5 is a flow diagram that illustrates the processing of the train demarcation machine learning model component of the OAW system in some embodiments. The train demarcation ML model component 500 trains a demarcation ML model to generate a demarcated patient-specific 3D mesh from a patient-specific 3D mesh and a demarcated generic 3D mesh. The demarcation ML model is trained using training sets that each includes a demarcated generic 3D mesh and a patient-specific 3D mesh labeled with a demarcated patient-specific 3D mesh. In block 501, the component selects the next training set. In decision block 502, if all the training sets have already been selected, then the component continues at block 510, else the component continues at block 503. In block 503, the component retrieves the cardiogram (ECG or VCG) of the training set. In block 504, the component retrieves the 3D image of the training set. In block 505, the component supplies the cardiogram to the mapping system which returns a demarcated generic 3D mesh with a source location demarcated. In block 506, the component applies a 3D ML model to the 3D image to generate a patient-specific 3D mesh with a segmentation of the 3D image. As described above, the 3D ML model may include a segmentation ML sub-model, a labeling ML sub-model, and a 3D sub-model. The segmentation ML sub-model may generate a segmentation of the heart and non-cardiac structures within the 3D image. Alternately, a separate segmentation ML model may be employed to segment the non-cardiac structure within the 3D image. In block 507, the component creates a feature vector that includes the patient-specific 3D mesh and the demarcated generic 3D mesh of the training set. In block 508, the component generates a label based on the demarcated patient-specific 3D mesh of the training set. In block 509, the component stores the feature vector and the label as training data in the training data store and loops to block 501 to select the next training set. In block 510, the component trains the demarcation ML model using the training data in the training data store and stores the model parameters (e.g., weights of activation functions and biases) of the demarcation ML model in the model parameter store. The other ML models are trained in a similar manner but with training sets as described above.

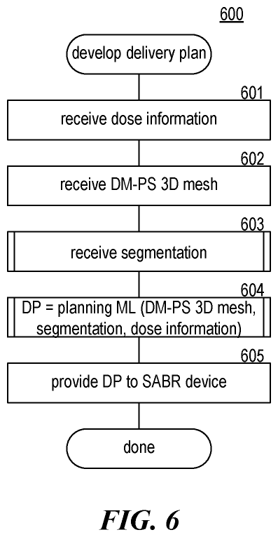

FIG. 6 is a flow diagram that illustrates the process of a develop delivery plan component of the OAW system in some embodiment. The develop delivery plan component 600 employs a planning ML model to develop a delivery plan for treating a patient. In block 601, the component receives dose information relating radiation for the ablation procedure such as maximum dosage for the target. In block 602, the component receives a demarcated patient-specific 3D mesh generated for the patient. In block 603, the component receives a segmentation of the patient's heart and non-cardiac structures within the patient's thorax. In block 604, the component applies a planning ML model to demarcated patient-specific 3D mesh, the segmentation, and dose information to generate the delivery plan (DP) for the patient. In block 605, the component provides the delivery plan to the SABR device and then completes.

The following paragraphs describe various embodiments of aspects of the OAW system and other system. An implementation of the systems may employ any combination of the embodiments. The processing described below may be performed by a computing system with a processor that executes computer-executable instructions stored on a computer-readable storage medium that implements the system.

In some aspects, the techniques described herein relate to a method performed by one or more computing systems for planning a cardiac stereotactic ablative radiotherapy procedure for a patient, the method including: accessing an arrhythmia electrocardiogram (ECG) of a patient, a 3D image of a thorax collected from the patient, and patient characteristics; applying a mapping system to the arrhythmia ECG and patient characteristics to generate a demarcated generic three-dimensional (3D) mesh of heart with a region of interest (ROI) demarcated; applying a 3D machine learning (ML) model to the 3D image to generate a patient-specific 3D mesh and a labeling of thoracic segments; applying a demarcation ML model to the patient-specific 3D mesh and the demarcated generic 3D mesh to generate a demarcated patient-specific 3D mesh; and generating a delivery plan for the patient by: identifying thoracic segments that represent avoidance structures; and applying a planning ML model to the demarcated patient-specific 3D mesh, avoidance structures, and radiation dosage information to generate a delivery plan for the stereotactic ablative radiotherapy procedure. In some aspects, the techniques described herein relate to a method wherein the delivery plan includes movements of a delivery arm of a stereotactic ablative radiotherapy device, doses of radiation for orientations of the delivery arm, and shapes of a delivery beam for the doses. In some aspects, the techniques described herein relate to a method further including sending the delivery plan to the stereotactic ablative radiotherapy device and directing the performing of the cardiac stereotactic ablative radiotherapy procedure according to the delivery plan. In some aspects, the techniques described herein relate to a method wherein a first ROI is a source location and a second ROI is scar tissue. In some aspects, the techniques described herein relate to a method wherein the mapping system includes a mapping ML model that inputs the arrhythmia ECG and patient characteristics and outputs the demarcated generic 3D mesh. In some aspects, the techniques described herein relate to a method wherein the avoidance structures are demarcated by a specification of a volume and location within the thorax of the patient. In some aspects, the techniques described herein relate to a method wherein the ROI is specified by a volume at a location within the demarcated patient-specific 3D mesh of a target of the stereotactic ablative radiotherapy procedure. In some aspects, the techniques described herein relate to a method wherein the 3D image is a computed tomography scan. In some aspects, the techniques described herein relate to a method wherein the 3D image is a magnetic resonance image scan.

In some aspects, the techniques described herein relate to a method for planning a procedure on an organ of a patient, the method including: applying a mapping system that inputs a patient electrogram representing electrical activity of the patient's organ and patient characteristics of the patient and outputs a demarcated generic three-dimensional (3D) mesh representing the organ based on a generic organ geometry and having one or more regions of interest (ROIs) within the demarcated generic 3D mesh demarcated, the one or more ROIs including a target region for the procedure; collecting a 3D image of at least a portion of the thorax of the patient that includes the patient's organ; generating a patient-specific 3D mesh representing the patient's organ based on the 3D image, the patient-specific 3D mesh representing patient organ geometry of the patient's organ; identifying segments within the 3D image and generating labels for the segments that indicate segment type; and applying a demarcation machine learning (ML) model to the patient-specific 3D mesh and the demarcated generic 3D mesh to generate a demarcated patient-specific 3D mesh with the one or more ROIs demarcated accounting for differences between the generic organ geometry and the patient organ geometry. In some aspects, the techniques described herein relate to a method further including generating a delivery plan for the patient based on the demarcated patient-specific 3D mesh, labels for the segments, and a radiation dosage information. In some aspects, the techniques described herein relate to a method wherein the mapping system applies a mapping ML model that inputs the patient electrogram and the patient characteristics and outputs the demarcated generic 3D mesh. In some aspects, the techniques described herein relate to a method wherein the mapping system identifies, from a library of associations between library electrograms and library demarcated generic 3D meshes, a library electrogram that matches the patient electrogram based on a matching criterion and outputs the library demarcated generic 3D mesh associated with the matching library electrogram. In some aspects, the techniques described herein relate to a method wherein the mapping system generates the demarcated generic 3D mesh based on a simulated organ geometry used in a simulation of electrical activity of the organ based on simulated organ characteristics that include the simulated organ geometry. In some aspects, the techniques described herein relate to a method further including applying a 3D ML model to the 3D image to generate the patient-specific 3D mesh, identify the segments, and generate the labels. In some aspects, the techniques described herein relate to a method wherein the organ is selected from the group consisting of a brain, a gastrointestinal organ, a heart, and a lung.

In some aspects, the techniques described herein relate to a method performed by one or more computing systems for generating a three-dimensional (3D) machine learning (ML) model, the method including: accessing training data that includes training sets that each includes features based on a 3D image that includes an organ with an organ geometry and based on characteristics associated with the organ and that each includes labels indicating a labeling of segments of the 3D image and indicating a 3D mesh that is based on that organ geometry; and training the 3D ML model based on the training sets, the 3D ML model for inputting features derived from a patient 3D image of the organ of the patient and characteristics associated with the patient's organ and outputting a labeling of the segments with the 3D image and a patient-specific 3D mesh representing the organ geometry of the patient. In some aspects, the techniques described herein relate to a method wherein the 3D ML model includes a segmentation ML sub-model that inputs the 3D image and outputs a segmentation of the 3D image, a labeling ML sub-model that inputs the segmentation of the 3D image and outputs a labeling of segments, and a 3D ML sub-model inputs the labeling of segments and outputs the patient-specific 3D mesh. In some aspects, the techniques described herein relate to a method wherein the organ is a heart. In some aspects, the techniques described herein relate to a method wherein at least some of the 3D images are collected using a scanning device and have a labeling of segments of the 3D image. In some aspects, the techniques described herein relate to a method wherein at least some of the 3D images are simulated 3D images, each simulated 3D image representing a different combination of segment geometries and segment positions of segments within a body. In some aspects, the techniques described herein relate to a method further including accessing a patient 3D image of the organ of a patient and patient characteristics and applying the 3D ML model to the patient 3D image and the patient characteristics to generate a patient-specific 3D mesh of the patient's organ and a labeling of segments within the patient 3D image.

In some aspects, the techniques described herein relate to a method performed by one or more computing systems for generating a planning machine learning (ML) model, the method including: accessing training data that includes training sets, each training set including features derived from a demarcated three-dimensional (3D) mesh representing an organ with a target region for a medical procedure demarcated and from other structures within a body, the features labeled with a delivery plan for the medical procedure; and training the planning ML model based on the training sets, the planning ML model for inputting features derived from a demarcated patient-specific 3D mesh representing the organ of a patient with a target region demarcated and from other structures within the patient's body and outputting a delivery plan for the medical procedure to treat the target region of the patient's organ. In some aspects, the techniques described herein relate to a method wherein the target region is demarcated using metadata associated with the demarcated 3D mesh. In some aspects, the techniques described herein relate to a method wherein a feature derived from the demarcated 3D mesh is a 3D image that includes the organ. In some aspects, the techniques described herein relate to a method wherein a feature is based on dosage information. In some aspects, the techniques described herein relate to a method wherein the organ is a heart and further including collecting a 3D image of a portion of the body of a patient that includes the heart and non-cardiac structures, generating features based on a demarcated patient-specific 3D mesh derived from the collected 3D image demarcated with a target region and a labeling of segments within the collected 3D image and applying the planning ML model to the features to generate a delivery plan for treating the patient.

In some aspects, the techniques described herein relate to a method performed by one or more computing systems for treating a patient by performing a cardiac stereotactic ablative radiotherapy procedure on the heart of the patient, the method including: collecting an arrhythmia electrocardiogram (ECG) and patient characteristics of the patient; receiving a demarcated generic three-dimensional (3D) mesh representing a generic cardiac geometry by inputting the arrhythmia ECG and patient characteristics into a mapping system that outputs the demarcated generic 3D mesh with a region of interest (ROI) demarcated; collecting a 3D image of the heart of the patient; generating based on the 3D image a patient-specific 3D mesh representing the patient's heart; and generating a demarcated patient-specific 3D mesh based on the patient-specific 3D mesh and the demarcated generic 3D mesh, the demarcated patient-specific 3D mesh with the ROI demarcated to reflect differences in the patient's cardiac geometry and the generic cardiac geometry. In some aspects, the techniques described herein relate to a method further including submitting the demarcated patient-specific 3D mesh to a stereotactic ablative radiotherapy device. In some aspects, the techniques described herein relate to a method further including generating a 3D image corresponding to the demarcated patient-specific 3D mesh and submitting the 3D image to a stereotactic ablative radiotherapy device. In some aspects, the techniques described herein relate to a method further including generating a labeling of segments within the 3D image and generating a delivery plan based on demarcated patient-specific 3D mesh, the labeled segments, and a target dose. In some aspects, the techniques described herein relate to a method wherein the delivery plan is generated using a planning machine learning model.

In some aspects, the techniques described herein relate to one or more computing systems for supporting treatment of a patient with an arrythmia, the one or more computing systems including: one or more computer-readable storage mediums that store: an arrhythmia cardiogram collected from the patient; a 3D image of the heart of the patient; and computer-executable instructions for controlling the one or more computing systems to: generate a demarcated generic three-dimensional (3D) mesh representing a generic cardiac geometry by inputting the arrhythmia cardiogram into a mapping system that outputs the demarcated generic 3D mesh with a region of interest (ROI) demarcated; generate based on the 3D image a patient-specific 3D mesh representing the patient's heart; and generate a demarcated patient-specific 3D mesh based on the patient-specific 3D mesh and the demarcated generic 3D mesh, the demarcated patient-specific 3D mesh with the ROI demarcated based on differences in the patient's cardiac geometry and the generic cardiac geometry; and one or more processors for controlling the one or more computing systems to execute one or more of the computer-executable instructions. In some aspects, the techniques described herein relate to one or more computing systems wherein the instructions that generate the patient-specific 3D mesh apply a 3D machine learning (ML) model that includes a segmentation ML sub-model, a labeling ML sub-model, and a 3D ML sub-model. In some aspects, the techniques described herein relate to one or more computing systems wherein the computer-executable instructions include instructions to generate a delivery plan based on the demarcated patient-specific 3D mesh, a labeling of segments within the patient's thorax, and dosage information. In some aspects, the techniques described herein relate to one or more computing systems wherein the computer-executable instructions include instructions to display a 3D graphic of a heart based on the patient-specific 3D mesh. In some aspects, the techniques described herein relate to one or more computing systems wherein the computer-executable instructions include instructions to generate a demarcated patient-specific 3D image based on the demarcated patient-specific 3D mesh. In some aspects, the techniques described herein relate to one or more computing systems wherein the computer-executable instructions include instructions to generate a demarcated patient-specific 3D image based on the demarcated patient-specific 3D mesh and the 3D image.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

I/We claim:

1. A method performed by one or more computing systems for planning a cardiac stereotactic ablative radiotherapy procedure for a patient, the method comprising:
accessing an arrhythmia electrocardiogram (ECG) of a patient, a 3D image of a thorax collected from the patient, and patient characteristics;
applying a mapping system to the arrhythmia ECG and patient characteristics to generate a demarcated generic three-dimensional (3D) mesh of heart with a region of interest (ROI) demarcated;
applying a 3D machine learning (ML) model to the 3D image to generate a patient-specific 3D mesh and a labeling of thoracic segments;
applying a demarcation ML model to the patient-specific 3D mesh and the demarcated generic 3D mesh to generate a demarcated patient-specific 3D mesh; and
generating a delivery plan for the patient by:
identifying thoracic segments that represent avoidance structures; and
applying a planning ML model to the demarcated patient-specific 3D mesh, avoidance structures, and radiation dosage information to generate a delivery plan for the stereotactic ablative radiotherapy procedure.

2. The method of claim 1 wherein the delivery plan includes movements of a delivery arm of a stereotactic ablative radiotherapy device, doses of radiation for orientations of the delivery arm, and shapes of a delivery beam for the doses.

3. The method of claim 2 further comprising sending the delivery plan to the stereotactic ablative radiotherapy device and directing the performing of the cardiac stereotactic ablative radiotherapy procedure according to the delivery plan.

4. The method of claim 1 wherein a first ROI is a source location and a second ROI is scar tissue.

5. The method of claim 1 wherein the mapping system includes a mapping ML model that inputs the arrhythmia ECG and patient characteristics and outputs the demarcated generic 3D mesh.

6. The method of claim 1 wherein the avoidance structures are demarcated by a specification of a volume and location within the thorax of the patient.

7. The method of claim 1 wherein the ROI is specified by a volume at a location within the demarcated patient-specific 3D mesh of a target of the stereotactic ablative radiotherapy procedure.

8. The method of claim 1 wherein the 3D image is a computed tomography scan.

9. The method of claim 1 wherein the 3D image is a magnetic resonance image scan.

10. A method for planning a procedure on an organ of a patient, the method comprising:
applying a mapping system that inputs a patient electrogram representing electrical activity of the patient's organ and patient characteristics of the patient and outputs a demarcated generic three-dimensional (3D) mesh representing the organ based on a generic organ geometry and having one or more regions of interest (ROIs) within the demarcated generic 3D mesh demarcated, the one or more ROIs including a target region for the procedure;
collecting a 3D image of at least a portion of the thorax of the patient that includes the patient's organ;
generating a patient-specific 3D mesh representing the patient's organ based on the 3D image, the patient-specific 3D mesh representing patient organ geometry of the patient's organ;
identifying segments within the 3D image and generating labels for the segments that indicate segment type; and
applying a demarcation machine learning (ML) model to the patient-specific 3D mesh and the demarcated generic 3D mesh to generate a demarcated patient-specific 3D mesh with the one or more ROIs demarcated accounting for differences between the generic organ geometry and the patient organ geometry.

11. The method of claim 10 further comprising generating a delivery plan for the patient based on the demarcated patient-specific 3D mesh, labels for the segments, and a radiation dosage information.

12. The method of claim 10 wherein the mapping system applies a mapping ML model that inputs the patient electrogram and the patient characteristics and outputs the demarcated generic 3D mesh.

13. The method of claim 10 wherein the mapping system identifies, from a library of associations between library electrograms and library demarcated generic 3D meshes, a library electrogram that matches the patient electrogram based on a matching criterion and outputs the library demarcated generic 3D mesh associated with the matching library electrogram.

14. The method of claim 10 wherein the mapping system generates the demarcated generic 3D mesh based on a simulated organ geometry used in a simulation of electrical activity of the organ based on simulated organ characteristics that include the simulated organ geometry.

15. The method of claim 10 further comprising applying a 3D ML model to the 3D image to generate the patient-specific 3D mesh, identify the segments, and generate the labels.

16. The method of claim 10 wherein the organ is selected from the group consisting of a brain, a gastrointestinal organ, a heart, and a lung.

17. A method performed by one or more computing systems for generating a three-dimensional (3D) machine learning (ML) model, the method comprising:
accessing training data that includes training sets that each includes features based on a 3D image that includes an organ with an organ geometry and based on characteristics associated with the organ and that each includes labels indicating a labeling of segments of the 3D image and indicating a 3D mesh that is based on that organ geometry; and
training the 3D ML model based on the training sets, the 3D ML model for inputting features derived from a patient 3D image of the organ of the patient and characteristics associated with the patient's organ and outputting a labeling of the segments with the 3D image and a patient-specific 3D mesh representing the organ geometry of the patient.

18. The method of claim 17 wherein the 3D ML model includes a segmentation ML sub-model that inputs the 3D image and outputs a segmentation of the 3D image, a labeling ML sub-model that inputs the segmentation of the 3D image and outputs a labeling of segments, and a 3D ML sub-model inputs the labeling of segments and outputs the patient-specific 3D mesh.

19. The method of claim 17 wherein the organ is a heart.

20. The method of claim 17 wherein at least some of the 3D images are collected using a scanning device and have a labeling of segments of the 3D image.

21. The method of claim 17 wherein at least some of the 3D images are simulated 3D images, each simulated 3D image representing a different combination of segment geometries and segment positions of segments within a body.

22. The method of claim 17 further comprising accessing a patient 3D image of the organ of a patient and patient characteristics and applying the 3D ML model to the patient 3D image and the patient characteristics to generate a patient-specific 3D mesh of the patient's organ and a labeling of segments within the patient 3D image.

23. A method performed by one or more computing systems for generating a planning machine learning (ML) model, the method comprising:
accessing training data that includes training sets, each training set including features derived from a demarcated three-dimensional (3D) mesh representing an organ with a target region for a medical procedure demarcated and from other structures within a body, the features labeled with a delivery plan for the medical procedure; and
training the planning ML model based on the training sets, the planning ML model for inputting features derived from a demarcated patient-specific 3D mesh representing the organ of a patient with a target region demarcated and from other structures within the patient's body and outputting a delivery plan for the medical procedure to treat the target region of the patient's organ.

24. The method of claim 23 wherein the target region is demarcated using metadata associated with the demarcated 3D mesh.

25. The method of claim 24 wherein a feature derived from the demarcated 3D mesh is a 3D image that includes the organ.

26. The method of claim 23 wherein a feature is based on dosage information.

27. The method of claim 24 wherein the organ is a heart and further comprising collecting a 3D image of a portion of the body of a patient that includes the heart and non-cardiac structures, generating features based on a demarcated patient-specific 3D mesh derived from the collected 3D image demarcated with a target region and a labeling of segments within the collected 3D image and applying the planning ML model to the features to generate a delivery plan for treating the patient.

28. A method performed by one or more computing systems for treating a patient by performing a cardiac stereotactic ablative radiotherapy procedure on the heart of the patient, the method comprising:
collecting an arrhythmia electrocardiogram (ECG) and patient characteristics of the patient;
receiving a demarcated generic three-dimensional (3D) mesh representing a generic cardiac geometry by inputting the arrhythmia ECG and patient characteristics into a mapping system that outputs the demarcated generic 3D mesh with a region of interest (ROI) demarcated;
collecting a 3D image of the heart of the patient;
generating based on the 3D image a patient-specific 3D mesh representing the patient's heart; and
generating a demarcated patient-specific 3D mesh based on the patient-specific 3D mesh and the demarcated generic 3D mesh, the demarcated patient-specific 3D mesh with the ROI demarcated to reflect differences in the patient's cardiac geometry and the generic cardiac geometry.

29. The method of claim 28 further comprising submitting the demarcated patient-specific 3D mesh to a stereotactic ablative radiotherapy device.

30. The method of claim 28 further comprising generating a 3D image corresponding to the demarcated patient-specific 3D mesh and submitting the 3D image to a stereotactic ablative radiotherapy device.

31. The method of claim 28 further comprising generating a labeling of segments within the 3D image and generating a delivery plan based on demarcated patient-specific 3D mesh, the labeled segments, and a target dose.

32. The method of claim 31 wherein the delivery plan is generated using a planning machine learning model.

33. One or more computing systems for supporting treatment of a patient with an arrythmia, the one or more computing systems comprising:
one or more computer-readable storage mediums that store:
an arrhythmia cardiogram collected from the patient;
a 3D image of the heart of the patient; and
computer-executable instructions for controlling the one or more computing systems to:
generate a demarcated generic three-dimensional (3D) mesh representing a generic cardiac geometry by inputting the arrhythmia cardiogram into a mapping system that outputs the demarcated generic 3D mesh with a region of interest (ROI) demarcated;
generate based on the 3D image a patient-specific 3D mesh representing the patient's heart; and
generate a demarcated patient-specific 3D mesh based on the patient-specific 3D mesh and the demarcated generic 3D mesh, the demarcated patient-specific 3D mesh with the ROI demarcated based on differences in the patient's cardiac geometry and the generic cardiac geometry; and
one or more processors for controlling the one or more computing systems to execute one or more of the computer-executable instructions.

34. The one or more computing systems of claim 33 wherein the instructions that generate the patient-specific 3D mesh apply a 3D machine learning (ML) model that includes a segmentation ML sub-model, a labeling ML sub-model, and a 3D ML sub-model.

35. The one or more computing systems of claim 33 wherein the computer-executable instructions include instructions to generate a delivery plan based on the demarcated patient-specific 3D mesh, a labeling of segments within the patient's thorax, and dosage information.

36. The one or more computing systems of claim 33 wherein the computer-executable instructions include instructions to display a 3D graphic of a heart based on the patient-specific 3D mesh.

37. The one or more computing systems of claim 33 wherein the computer-executable instructions include instructions to generate a demarcated patient-specific 3D image based on the demarcated patient-specific 3D mesh.

38. The one or more computing systems of claim 33 wherein the computer-executable instructions include instructions to generate a demarcated patient-specific 3D image based on the demarcated patient-specific 3D mesh and the 3D image.
